(19) United States
(12) Patent Application Publication
(10) Pub. No.: US 2006/0004284 A1
(43) Pub. Date: Jan. 5, 2006

(54) Method and system for generating three-dimensional model of part of a body from fluoroscopy image data and specific landmarks

(76) Inventors: Frank Grunschlager, Feldkirchen (DE); Martin Haimerl, Gilching (DE); Rainer Lachner, Poing (DE); Stefan Vilsmeier, Kufstein (AT); Alf Ritter, Kleinberghofen (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 11/171,122

(22) Filed: Jun. 30, 2005

Related U.S. Application Data
(60) Provisional application No. 60/588,898, filed on Jul. 16, 2004.

(30) Foreign Application Priority Data
Jun. 30, 2004 (EP) 04015308.2

(51) Int. Cl. A61B 5/05

(52) U.S. Cl. 600/416

(57) ABSTRACT

A method for generating a three-dimensional model of a part of the body with the aid of a medical and/or surgical navigation system, comprising the steps of identifying to the navigation system landmarks on the part of the body that are characteristic of the model of the part of the body; obtaining at least two fluoroscopy image data sets for each of one or more predetermined, individual and delimited regions of the part of the body; ascertaining characteristic body part data by processing and combining the landmark positions and parameters of the fluoroscopy data sets; and generating a three-dimensional and positionally determined model of the part of the body from the characteristic body part data.

### RELATED APPLICATION DATA

This application claims priority of U.S. Provisional Application No. 60/588,898 filed on Jul. 16, 2004, which is hereby incorporated herein by reference in its entirety.

### FIELD OF THE INVENTION

The present invention relates generally to a method for generating a three-dimensional model of a part of a body with the aid of a medical and/or surgical navigation system and, more particularly, to generating such a model without preceding tomographic imaging.

### BACKGROUND OF THE INVENTION

Currently used techniques for computer tomographic-free navigation mainly involve navigation with the aid of x-ray images obtained from a fluoroscopy apparatus. Using the fluoroscopy apparatus, images are acquired and, after calibration and distortion correction, landmarks are determined in the images. This purely fluoroscopic navigation is awkward and often requires many x-ray recordings in order to obtain all the necessary data available at any desired point in time. Additionally, the numerous x-ray recordings cause an increased radiation load on the patient and on the operating staff.

European Patent No. EP 1 329 202 B1 describes a method and apparatus for assigning digital image information to navigation data of a medical navigation system, wherein image data produced using a digital C-arc x-ray apparatus are incorporated into navigation. Using this technique, numerous x-ray recordings are taken in succession, which, like above, causes a corresponding radiation load on the patient and staff.

### SUMMARY OF THE INVENTION

The present invention provides a method for generating a three-dimensional model of a part of the body that overcomes disadvantages of the prior art. In particular, the invention enables three-dimensional navigation using simple means without acquiring tomographs, specifically computer tomograph (CT) recordings, prior to performing the navigation. The invention can produce a three-dimensional model of a part of the body, thereby permitting navigation without successively obtaining new fluoroscopy recordings.

A method in accordance with the invention uses fluoroscopy image data sets in conjunction with positional identification of characteristic landmarks on a body part to generate a model of the body part. Combining fluoroscopy with positional identification of characteristic landmarks to generate the model of the part of the body results in much simpler and less elaborate calculations than using fluoroscopy image data sets alone.

The identified landmarks reproduce absolute spatial points which, in conjunction with fluoroscopic data, facilitate production of the model. In other words, the present invention combines two methods of detecting body features, each of which can be performed independently, in such a way so as to produce a three-dimensional model.

In accordance with the invention, two fluoroscopy image data sets obtained from different detection directions for each of particular, individual and delimited region of a part of the body, are used to produce the three-dimensional model. In addition, the positional data acquired from each technique can supplement data obtained from the other technique. For example, points that cannot be tapped by a pointer can be determined from fluoroscopic transillumination images by performing a symmetry calculation on symmetrically or substantially symmetrically formed parts of the body.

The characteristic body part data can include lengths of body part sections and angles of body part sections with respect to each other. In accordance with the invention, joint rotation center points also can be determined by positionally identifying characteristic landmarks (or a navigation reference array, such as a reference star, on a movable joint bone) at a number of angular positions of the joint and then calculating back to the rotational center from the obtained trajectory points.

The part of the body to be modeled may be the femur, pelvis, etc.

The model of the part of the body may be supplemented or completed with the aid of generic body part data, in particular by using a generic model of the part of the body that has been adapted on the basis of information already ascertained for the model of the part of the body to be generated.

An image output may display, before a landmark is identified or the fluoroscopy image data sets are produced, which landmark is to be identified (e.g., tapped with a pointer) next in succession and/or which fluoroscopy image is to be obtained next in succession.

Accordingly, a method for generating a three-dimensional model of a part of the body with the aid of a medical and/or surgical navigation comprises the steps of identifying to the navigation system landmarks on the part of the body that are characteristic of the model of the part of the body, obtaining at least two fluoroscopy image data sets for each of one or more predetermined, individual and delimited regions of the part of the body, and ascertaining characteristic body part data by processing and combining the landmark positions and parameters of the fluoroscopy data sets. A three-dimensional and positionally determined model of the part of the body then may be generated from the characteristic body part data.

Further features of the invention will become apparent from the following detailed description when considered in conjunction with the drawings.

### BRIEF DESCRIPTION OF THE DRAWINGS

FIG. 1 is a schematic representation of a femur and pelvis.

FIG. 2 is a schematic representation in accordance with FIG. 1, additionally displaying regions for fluoroscopy recordings in accordance with an embodiment of the invention.

FIG. 3 is a screen shot of a program for assisting in recording fluoroscopy image data sets in accordance with an embodiment of the invention.

FIG. 4 is a block diagram of a computer system that can be used to implement the method of the present invention.

### DETAILED DESCRIPTION

The invention will now be described in more detail on the basis of generating a model that can be used in hip operations. It should be appreciated, however, that the present invention can be applied to other medical procedures, and reference to hip operations is not intended to be limiting in anyway.

The invention provides a navigation method based on a three-dimensional model generated from two-dimensional fluoroscopic image recordings and specific landmarks of a part of the body. As a result, the three-dimensional orientation of the human pelvis is improved with respect to prior techniques, thereby enabling an implant to be suitably positioned in surgical total hip replacement procedures. This includes not only the hip bone but also the femur.

Two orientation parameters are important when positioning a cavity implant: the cavity anteversion and the cavity inclination. These two parameters relate to a hip coordinate system that is defined by the anatomy of the hip bone, i.e., by a frontal pelvic plane and a mid-sagittal plane. These two planes represent the basis for all angular calculations used to place the cavity implant in replacement of the anatomical hip joint.

Two relevant orientation parameters also are defined for placing a femur implant, namely, the shaft axis and the neck axis (neck axis of the femur). These two axes represent the basis for all calculations for placing the shaft implant and/or the femur implant when replacing the anatomical hip joint.

In accordance with the present invention, specific landmarks may be acquired using a navigation pointer of a medical navigation system, and information relating to the landmarks is combined with information obtained from fluoroscopy images. The medical navigation system can include a navigation system, such as is described in co-owned U.S. Pat. No. 6,351,659, which is incorporated herein by reference in its entirety. The navigation pointer includes reference elements, which allow the navigation system to track the location of the pointer within a medical workspace.

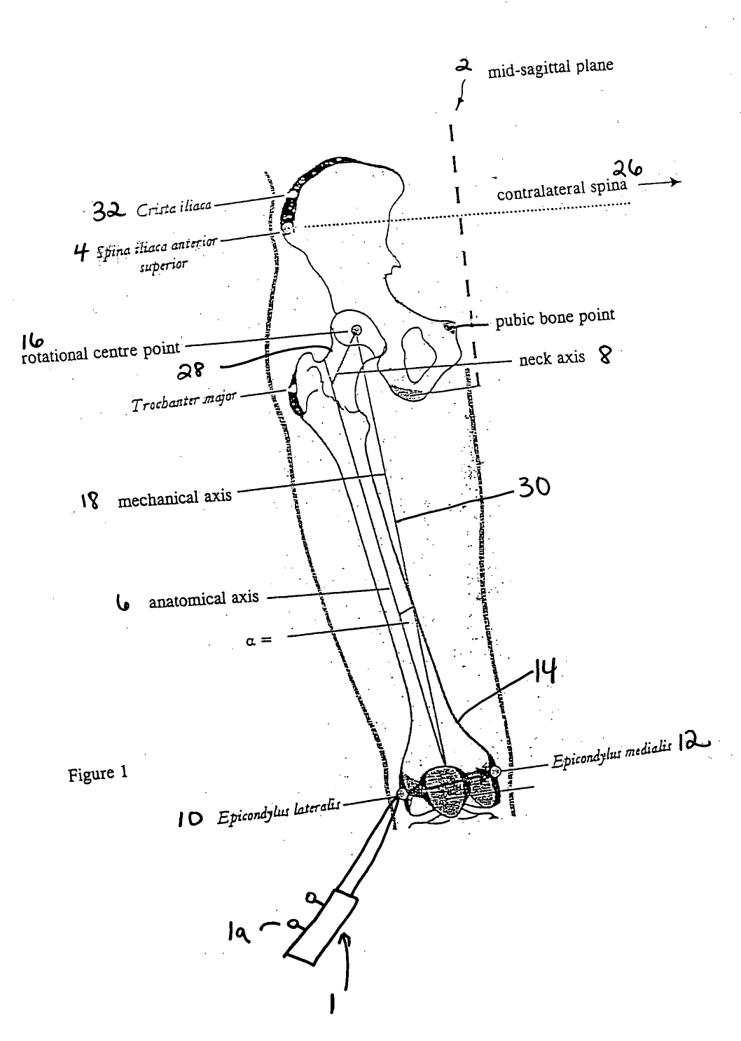

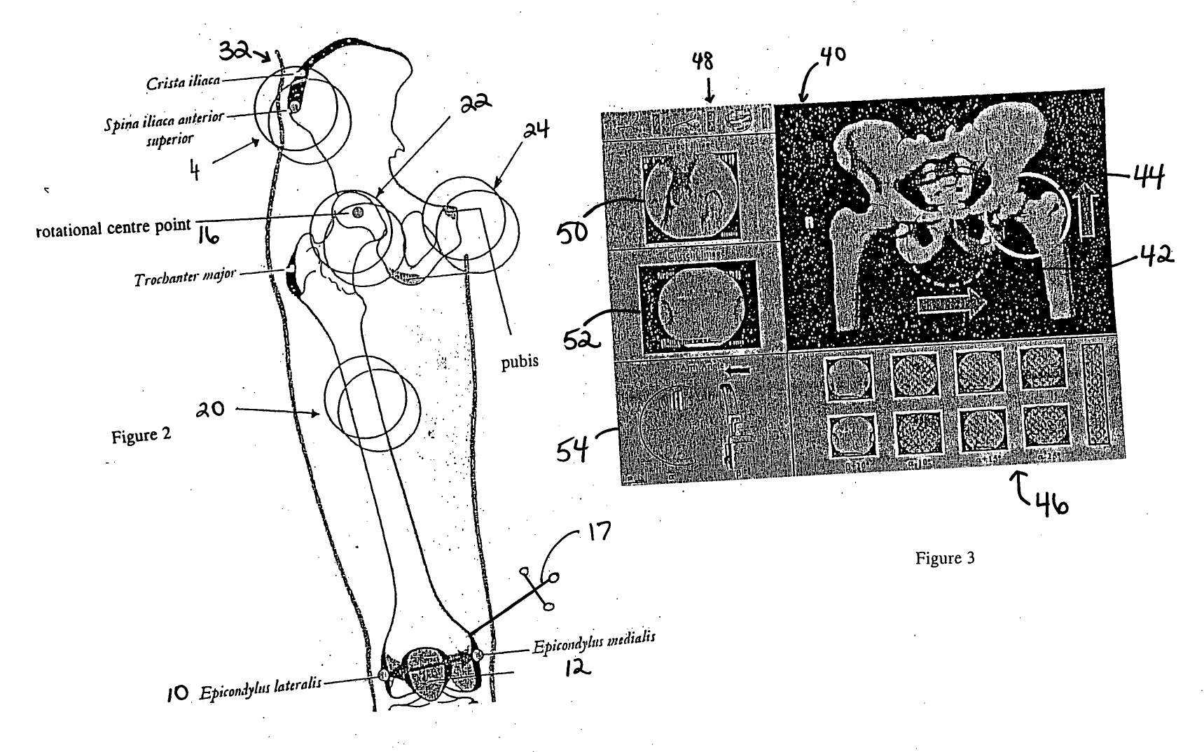

The fluoroscopy images can be obtained using image processing algorithms, for example. As will be described in more detail below, FIG. 1 illustrates points that are acquired using a navigation pointer 1, such as the spina iliaca anterior superior, the epicondylus lateralis and the epicondylis medialis. The navigation pointer 1 includes reference markers 1a, which permit the pointer 1 to be tracked by the medical navigation system. FIG. 2 indicates regions 20-26 for which fluoroscopy recordings are obtained, wherein the two circles shown for each region indicate that two fluoroscopy recordings are obtained from different angles for each region. The two images for each region enable the detected features to be three-dimensionally reconstructed. Image processing then can be performed either by the fluoroscopy apparatus itself or via an image processing unit of the medical navigation system. As used herein, image processing is defined as the computer manipulation of images using one or more image processing algorithms, including, but not limited to, convolution, Fast Fourier Transform, Discrete Cosine Transform, thinning (or skeletonization), edge detection and contrast enhancement.

Determining the coordinate system of the pelvis is difficult when specific landmarks are not physically acquired or acquirable. In the absence of landmarks, it is necessary to fall back on fluoroscopy images that describe the bone structure (pelvis or femur). Symmetry of the bone structures can be utilized here. For example, the mid-sagittal plane 2 of the pelvis can be determined without the navigation pointer 1 having access to both spina iliaca anterior superior 4 points. The mid-sagittal plane 2 can be determined by obtaining x-ray recordings of the tuberculum pubis region. An automatic algorithm then can calculate the image's center axis of symmetry, and another automatic algorithm can calculate the orientation of the center axis of symmetry. This is likewise possible for the femur shaft axis 6 and the neck axis 8.

A spina iliaca anterior superior point 4 usually can be tapped relatively simply using the navigation pointer 1. This also applies to the epicondylus lateralis 10 and the epicondylus medialis 12 on the femur 14. As used herein, to tap or tapping a landmark refers to positioning one end of a trackable pointer 1 substantially on the landmark such that the landmark position can be recorded by the medical navigation system.

The rotational center point 16 for the femur can be calculated by positionally tracking the landmarks 4, 10, 12 or by tracking a reference array 17 (FIG. 2, e.g., a reference star) on the bone as the bone is pivoted in the medical navigation system. As the bone is pivoted, the landmarks move on a spherical surface and the center point of this surface defines the rotational center point of the femur. Using software, a mechanical axis 18, which is determined by two points, e.g., by the rotational center point and the center of the epicondular axis, then can be defined.

Once this pre-registration has been completed, eight fluoroscopy images, e.g., four pairs of fluoroscopy images in specific and delimited regions, are produced as indicated in FIG. 2 by the reference numerals 20 to 26. The specific regions for the fluoroscopy images are: the proximal femur 20; the neck of the femur 22; the pubis 24; and the spina iliaca anterior superior 26. Two fluoroscopy images are made for each region in order to obtain three-dimensional information from the two-dimensional images.

The image information from the pubic region allows, among other things, the mid-sagittal plane 2 to be ascertained, the contralateral spina point 26 to be calculated, and the frontal pelvic plane (not shown) to be ascertained. For example, the frontal pelvic plane can be defined by a pubic bone point and both spina points 4 and 26. The pubic bone point also can be calculated from the images.

The image information for the acetabulum/neck of the femur allows, among other things, the neck axis 8 to be ascertained by means of active contours, a predetermined value for the neck axis to be used on the basis of the angle between the neck 28 and the shaft 30 (about 130°), the head of the femur to be automatically ascertained (the size of the cavity equals the diameter of the head plus eight millimeters), and a leg length (LL) calculation from the position of the cavity and the femur implant.

The neck axis can be determined in a manner similar to that of the shaft axis (discussed below). Since the shaft axis is already known, as well as an estimation of the center of rotation of the shaft axis, a rough estimation of the location of the neck contours can be identified in the images. The two neck contours and the “round” portion of the femur head can be determined in both images (e.g., by active contours of the images in FIGS. 1 and 2). This information can be propagated to three dimensions, thus yielding the neck axis, improved center of rotation and the size of the femur head.

The image data for the proximal femur enable, among other things, the femur shaft axis 6 to be detected (the anatomical axis by means of active contours). The angle between the anatomical axis 6 and mechanical axis 18 of the femur can be presupposed to be 7° and, using this information, the size of the femur implant can be determined.

For example, a center of rotation of the femur can be accurately determined (e.g., within 3 mm) by pivoting the femur about the rotational center point 16, while points on the medial and lateral condyle 10, 12 can be determined via a pointer. The mechanical axis 18 runs through center of rotation and mid-point of the condyle points. Utilizing the 7° assumption, the direction of the shaft axis is roughly known (e.g., within 5°), and the bone contours of the shaft are detected in both images (e.g., using an active contour method of the images in FIGS. 1 and 2). The direction of these contours is similar to the shaft axis (which is roughly known). Hence, contour detection is very stable since the “search region” is quite small. The shaft axis can be determined as the line that is most “central” to the two contours (in the least squares sense). This is done in both images. The two dimensional axes are back projected to three dimensions, thus creating three dimensional planes. The three dimensional shaft axis is at the intersection of these planes.

The image information in the region of the spina iliaca anterior superior 4 allows, among other things, the crista iliaca 32 to be automatically detected. The contour of the crista iliaca, for example, can be identified in both images (FIGS. 1 and 2). Again, an active contour method can be used to automatically find the contours in the images (the position and orientation is already roughly known). This provides two two-dimensional curves, which are back projected to three dimensions to obtain two surfaces. The intersection of the two surfaces yields the three dimensional curve of the crista iliaca.

Using the information thus obtained, a three-dimensional model for the pelvis and femur can be defined, said model enabling navigation and allowing planning of implant placement.

Correctly acquiring the fluoroscopy images can be simplified via a software assistant. The software assistant guides the operating team through the acquisition of fluoroscopy images, and is sub-divided into various sections. The main part of the assistant includes a model with two circles, which is shown in the screen shot 40 in FIG. 3. The broken circle 42 describes the actual position of the C-arc fluoroscopy apparatus which for this purpose can be tracked by the medical navigation system. The continuous circle 44 shows the target position for producing a fluoroscopy image of a specific region. In the lower part 46 of the display 40, a status indicator displays which images have already been acquired and which are still to be produced.

At the top left of the display 40, reference arrays 48 relating to the software tools are displayed using color markings. The reference arrays assist the user in setting the angle from which the images will be obtained. A target image 50 helps find the best image for a specific region. These images can be compared to the then current image 52 shown below the target image. The final sector 54, bottom left, displays the angle of the C-arc.

With the aid of such an assistant, acquiring the fluoroscopy images is made simple and quick. Since the positions of the landmarks also can be acquired quickly via the pointer, a three-dimensional model of a part of the body, according to the invention, can be generated in a simple and quick way. The model can be used to plan and/or navigate surgical instruments before and during a surgical procedure. Alternatively, the model can provide supplemental data to the surgeon.

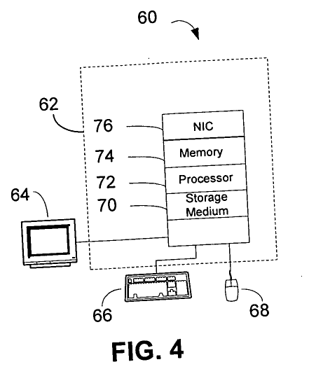

Moving to FIG. 4, a computer system 60 for executing a computer program in accordance with the present invention is illustrated. The computer system 60 includes a computer 62 for processing data, and a display 64 for viewing system information. The technology used in the display is not critical and may be any type currently available, such as a flat panel liquid crystal display (LCD) or a cathode ray tube (CRT) display, or any display subsequently developed. A keyboard 66 and pointing device 68 may be used for data entry, data display, screen navigation, etc. The keyboard 66 and pointing device 68 may be separate from the computer 62 or they may be integral to it. A computer mouse or other device that points to or otherwise identifies a location, action, etc., e.g., by a point and click method or some other method, are examples of a pointing device. Alternatively, a touch screen (not shown) may be used in place of the keyboard 66 and pointing device 68. A touch screen is well known by those skilled in the art and will not be described herein.

Included in the computer 62 is a storage medium 70 for storing information, such as application data, screen information, programs, etc. The storage medium 70 may be a hard drive, for example. A processor 72, such as an AMD Athlon 64™ processor or an Intel Pentium IV® processor, combined with a memory 74 and the storage medium 70 execute programs to perform various functions, such as data entry, numerical calculations, screen display, system setup, etc. A network interface card (NIC) 76 allows the computer 62 to communicate with devices external to the computer system 60.

The actual code for performing the functions described herein can be readily programmed by a person having ordinary skill in the art of computer programming in any of a number of conventional programming languages based on the disclosure herein. Consequently, further detail as to the particular code itself has been omitted for sake of brevity. As will be appreciated, the various computer codes for carrying out the processes herein described can be embodied in computer-readable media.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for generating a three-dimensional model of a part of the body with the aid of a medical navigation system, comprising:
identifying to the navigation system landmarks on the part of the body that are characteristic of the model of the part of the body;
obtaining at least two fluoroscopy image data sets for each of one or more predetermined, individual and delimited regions of the part of the body;
ascertaining characteristic body part data by processing and combining the landmark positions and parameters of the fluoroscopy data sets; and
generating a three-dimensional and positionally determined model of the part of the body from the characteristic body part data.

2. The method of claim 1, wherein identifying to the navigation system landmarks includes using a navigated pointer to indicate a position of each landmark.

3. The method of claim 1, further comprising image processing of the fluoroscopy data sets to obtain said parameters.

4. The method of claim 1, wherein ascertaining characteristic body part data includes using other landmarks obtained from a symmetry calculation on substantially symmetrical parts of the body.

5. The method of claim 1, wherein the characteristic body part data include lengths of body part sections and angles of body part sections with respect to each other.

6. The method of claim 1, further comprising determining joint rotation center points by positionally determining characteristic landmarks on the part of the body; and calculating a rotational center from a trajectory of the characteristic landmarks as the part of the body is rotated about an axis.

7. The method of claim 1, further comprising determining joint rotation center points by positionally determining a navigation reference array fixed to the part of the body; and calculating a rotational center from a trajectory of the reference array as the part of the body is rotated about an axis.

8. The method of claim 1, wherein the part of the body is a femur, further comprising:
determining a position of at least one of an epicondylus lateralis or an epicondylus medialis;
determining a joint rotation center point of the femur, including at least one of:
image processing using fluoroscopy images recorded in various joint angular positions, and
positionally determining one of the epicondylus lateralis, the epicondylus medialis, or a navigation reference array at a number of joint angular positions thereby creating a plurality of trajectory points and calculating back to a center point from the obtained trajectory points;
determining a mechanical axis of the femur by connecting the joint rotation center point and a center point between the epicondylus lateralis and epicondylus medialis;
determining an anatomical axis of the femur by image processing using fluoroscopy images of a bone shaft region of the femur, wherein a number of axis points in a center of the bone are determined which then determine the course of the anatomical axis;
determining a neck axis by at least one of:
image processing using two fluoroscopy images of the region of the neck of the femur,
image processing using one fluoroscopy image of the region of the neck of the femur and using the position of the joint rotation center point and a known and/or predetermined skewed position of the neck axis relative to the anatomical axis, and
tapping and positionally determining points and/or center points on the neck of the femur; and
determining antetorsion from the position of the mechanical axis and a position of a connection between the epicondylus lateralis and epicondylus medialis.

9. The method of claim 8, further comprising using a navigated pointer to indicate the position of at least one of the epicondylus lateralis or the epicondylus medialis.

10. The method of claim 1, wherein the part of the body is a pelvis, further comprising:
determining a position of at least one spina iliaca anterior superior;
determining a mid-sagittal plane of the pelvis by image processing using fluoroscopy images of a symmetrical pelvic structure region and the symmetry properties of the pelvis; and
determining a frontal pelvic plane by ascertaining a position of a front point of a pubic bone by image processing using fluoroscopy images of a pubic symphysis region and by connecting the front point to the position ascertained for the spina iliaca anterior superior.

11. The method of claim 10, wherein determining the mid-sagittal plane further includes ascertaining a position of a second spina iliaca anterior superior.

12. The method of claim 10, further comprising using a navigated pointer to indicate the position of the at least one spina iliaca anterior superior.

13. The method of claim 1, further comprising supplementing the model of the part of the body with generic body part data.

14. The method of claim 13, wherein supplementing the body part data further includes using a generic model of the part of the body which has been adapted on the basis of information already ascertained for the model of the part of the body to be generated.

15. The method of claim 1, further comprising displaying an image output, wherein prior to tapping each landmark or obtaining the fluoroscopy image data sets, the displayed image indicates which landmark is to be tapped next in succession and/or which fluoroscopy image is to be produced next in succession.

16. A system for generating a three-dimensional model of a part of the body with the aid of a medical navigation system, comprising:
a processor circuit having a processor and a memory;
a model generation subsystem stored in the memory and executable by the processor, the subsystem comprising:
logic that directs the identification to the navigation system landmarks on the part of the body that are characteristic of the model of the part of the body;
logic that obtains at least two fluoroscopy image data sets for each of one or more predetermined, individual and delimited regions of the part of the body;
logic that ascertains characteristic body part data by processing and combining the landmark positions and parameters of the fluoroscopy data sets; and
logic that generates a three-dimensional and positionally determined model of the part of the body from the characteristic body part data.

17. A program embodied in a computer-readable medium for generating a three-dimensional model of a part of the body with the aid of a medical navigation system, comprising:
code that directs the identification to the navigation system landmarks on the part of the body that are characteristic of the model of the part of the body;
code that obtains at least two fluoroscopy image data sets for each of one or more predetermined, individual and delimited regions of the part of the body;
code that ascertains characteristic body part data by processing and combining the landmark positions and parameters of the fluoroscopy data sets; and
code that generates a three-dimensional and positionally determined model of the part of the body from the characteristic body part data.
